(19) United States
(12) Patent Application Publication
(10) Pub. No.: US 2019/0201035 A1
(43) Pub. Date: Jul. 4, 2019

(54) PRECISION BLADE APPARATUS

(71) Applicant: Farid Amirouche, Highland Park, IL (US)

(72) Inventor: Farid Amirouche, Highland Park, IL (US)

(21) Appl. No.: 16/237,449

(22) Filed: Dec. 31, 2018

Related U.S. Application Data
(60) Provisional application No. 62/612,606, filed on Dec. 31, 2017.

(51) Int. Cl. A61B 17/32; A61B 17/3211

(52) U.S. Cl. CPC A61B 17/320016; A61B 1/00087; A61B 2017/32113; A61B 17/3211

(57) ABSTRACT

A precision blade apparatus includes a housing, a control mechanism, a blade motor and a power source, an adjustment dial, a retractable interchangeable blade operably coupled to the blade motor and configured to be operated by the control mechanism, an alignment mechanism operated by the control mechanism, and a secondary alignment mechanism attached to the alignment mechanism. The secondary alignment mechanism is configured to be aligned into a single point when the target object is appropriately centered within the alignment mechanism thereby allowing more nuanced control and illumination of a particular area of the target object. The adjustment dial is configured to calibrate and adjust a blade extension speed, a blade extension distance, a blade selection of the retractable interchangeable blade, a position of the alignment mechanism, operation of the secondary alignment mechanism, and power consumption.

### CROSS REFERENCE TO RELATED APPLICATIONS

This is a non-provisional patent application that claims the benefit of co-pending U.S. provisional patent application No. 62/612,606 filed Dec. 31, 2017, which is incorporated by reference herein in its entirety.

### STATEMENT REGARDING FEDERALLY SPONSORED RESEARCH OR DEVELOPMENT

Not Applicable.

### REFERENCE TO A MICROFICHE APPENDIX

Not Applicable.

### BACKGROUND

### Technical Field

Exemplary embodiment(s) of the present disclosure relate to surgical tools and, more particularly, to a blade apparatus including a streamlined grip and cutting tool that provides a surgeon greater control and precision during a surgical procedure.

### Prior Art

Surgery involves the cutting of a patient's tissues or closure of a previously sustained wound. Surgery is typically an invasive procedure, though sometimes excisions may be made that do not penetrate whatever is being excised from a patient. Surgical procedures may require a sterile environment, use surgical instruments, or involve suturing or stapling. Surgical instruments include tools specifically designed for performing surgery and are broken into categories based on their functionality, such as graspers, clamps, dilators, or retractors.

Medical procedures depend heavily on the ability of surgeons to operate with a steady hand, using sharp but often rudimentary tools. During an operation or surgical procedure, a trained surgeon uses various tools to manipulate the body's tissues and organs. While certain surgical tools, such as a scalpel or clamp, are used for most operative procedures, others are more specialized and may be used only with certain types of surgery. As medical procedures became more advanced and sophisticated, so too did some of the surgical tools.

In some circumstances, the use of advanced robotics allowed for some automation of the procedures in conjunction with the expertise and experience of surgeons. Still, for the majority of procedures, the preference is to allow surgeons the primary control over procedures, which is particularly important for uncommon procedures where surgeons may be required to make emergency decisions throughout the procedure.

Despite the advancement of many surgical procedures, many operations still utilize rudimentary tools, especially for the steps that require cutting. Accuracy and continuity during surgery are important parameters for the success of a surgical intervention, which places a high demand on the surgical instrument that is used. This dependency on basic cutting tools increases the risk for mistakes and reduces the ability of surgeons to operate with nuanced precision during sophisticated and lengthy procedures.

Some surgical procedures require extensive precision and still depend on rudimentary tools that are used for less sophisticated procedures. For example, procedures that involve soft tissue, nerves, removal of a tumor, or cartilage require precise incisions. Further, complex procedures may benefit from real-time feedback and information. This may allow for increased procedural awareness during and after a procedure.

What is needed is a surgical tool that may allow surgeons to have more control and accuracy for cutting during procedures. Accordingly, the present disclosure provides generally for a surgical tool with increased precision and easy handling through preset, manual, or automated means. More specifically, the present disclosure relates to a blade apparatus that may allow a surgeon greater control and precision during a procedure.

### BRIEF SUMMARY OF NON-LIMITING EXEMPLARY EMBODIMENT(S) OF THE PRESENT DISCLOSURE

In view of the foregoing background, it is therefore an object of the non-limiting exemplary embodiment(s) to provide a precision blade apparatus for precisely severing a target object including at least one of a vein, an artery, a capillary, a nerve muscle, and an organ during a medical surgical procedure. These and other objects, features, and advantages of the non-limiting exemplary embodiment(s) are provided by a precision blade apparatus including a housing, a control mechanism attached to the housing, a blade motor and a power source each in communication with the control mechanism and contained within the housing, an adjustment dial located at a proximal end of the housing and being operably coupled to the blade motor, a retractable interchangeable blade operably coupled to the blade motor and configured to be operated by the control mechanism and extended outwardly from a distal end of the housing, an alignment mechanism attached to an outer surface of the housing and operated by the control mechanism, and a secondary alignment mechanism attached to the alignment mechanism. Such a secondary alignment mechanism includes a series of light-emitting diodes configured to be aligned into a single point when the target object is appropriately centered within the alignment mechanism thereby allowing more nuanced control and illumination of a particular area of the target object. Advantageously, the adjustment dial is configured to calibrate and adjust a blade extension speed, a blade extension distance, a blade selection of the retractable interchangeable blade, a position of the alignment mechanism, operation of the secondary alignment mechanism, and power consumption.

The present disclosure further includes a precision blade apparatus for precisely severing a target object including at least one of a vein, an artery, a capillary, a nerve muscle, and an organ during a medical surgical procedure. Such a precision blade apparatus includes a portable, hand-held housing, a control mechanism attached to the housing, a blade motor and a power source each in communication with the control mechanism and contained within the housing, an adjustment dial located at a proximal end of the housing and being operably coupled to the blade motor, a retractable interchangeable blade operably coupled to the blade motor and configured to be operated by the control mechanism and extended outwardly from a distal end of the housing, an alignment mechanism attached to an outer surface of the housing and operated by the control mechanism, and a secondary alignment mechanism attached to the alignment mechanism. Such a secondary alignment mechanism including a series of light-emitting diodes configured to be aligned into a single point when the target object is appropriately centered within the alignment mechanism thereby allowing more nuanced control and illumination of a particular area of the target object. Advantageously, the adjustment dial is configured to calibrate and adjust a blade extension speed, a blade extension distance, a blade selection of the retractable interchangeable blade, a position of the alignment mechanism, operation of the secondary alignment mechanism, and power consumption.

In a non-limiting exemplary embodiment, the retractable interchangeable blade includes a plurality of uniquely sized interchangeable retractable blades.

In a non-limiting exemplary embodiment, the housing further includes a gripping mechanism including finger divots configured to limit movement of a user hand. Such a gripping mechanism includes a textured surface and an anti-slippage material.

In a non-limiting exemplary embodiment, the alignment mechanism includes a curving arm configured to accurately and precisely align the retractable blade with the target object. Such an alignment mechanism is configured to collect and stabilize the target object.

In a non-limiting exemplary embodiment, the control mechanism includes a plurality of buttons configured to maintain control over the blade extension and nuanced control over the blade extension speed within a predefined range based on a predetermined base blade extension speed.

In a non-limiting exemplary embodiment, one of the buttons is configured to trigger the blade motor to extend and retract the retractable interchangeable blade.

In a non-limiting exemplary embodiment, a blade pocket located at the distal end of the housing, and a blade insertion mechanism located at the distal end of the housing and configured to receive the retractable interchangeable blade proximate to the blade pocket such that the blade insertion mechanism grabs, accepts, and ejects the retractable interchangeable blade relative to the blade pocket.

In a non-limiting exemplary embodiment, the alignment mechanism is configured to be toggled between a released position and an engaged position such that the alignment mechanism is detachably and interchangeably attached to an exterior of the housing.

In a non-limiting exemplary embodiment, the blade pocket includes a plurality of tapering walls configured to limit movement of the retractable interchangeable blade when secured within the blade pocket.

In a non-limiting exemplary embodiment, a pivot control mechanism operably coupled to the control mechanism, a removable head housing operably coupled to the pivot control mechanism, and a pivoting neck operably coupled to the pivot control mechanism to pivot to a desired cut angle without straining a user hand. Notably, the removable head housing is configured to be slipped over the alignment mechanism once the target object is collected and before the cutting procedure. In this manner, the removable head housing is configured to coil around the target object.

In a non-limiting exemplary embodiment, the removable head housing includes a plurality of walls prefilled with environment control chemicals that are periodically released during the medical surgical procedure to lower an environmental temperature and slow blood flow at the target object, a plurality of solid-state thermal plates configured to precisely control the environmental temperature at the target object, and an environment control tube configured to pump in the environment control chemicals throughout the medical surgical procedure and thereby lower the environmental temperature at the target object.

In a non-limiting exemplary embodiment, the environment control chemicals includes: carbon dioxide, nitrogen, and nitric oxide configured to flow through the removable head housing. Advantageously, the alignment mechanism and the removable head housing are configured to control a temperature of a surface of the retractable interchangeable blade. In this manner, the pivoting neck is connected to the removable head housing, and the removable head housing is configured to surround the target object, the alignment mechanism, the retractable interchangeable blade, and the blade pocket.

In a non-limiting exemplary embodiment, the pivoting neck is configured to adjust an angle of the removable head housing relative to the gripping mechanism. Notably, the angle of the pivoting neck is operably coupled to the pivot control mechanism located adjacent to the control mechanism.

In a non-limiting exemplary embodiment, the alignment mechanism includes an external guide internal guide slidably secured thereto. Advantageously, the secondary alignment mechanism is attached to the internal guide. Notably, each of the external guide and the internal guide are configured to be directly controlled at the housing. In this manner, the alignment mechanism is configured to be retracted and collapsed around a surface opening of the blade pocket as well as the retractable interchangeable blade during non-use conditions.

In a non-limiting exemplary embodiment, the retractable interchangeable blade is configured to be selectively extended outwardly from the blade pocket and configured to be operated by the control mechanism. Advantageously, the retractable interchangeable blade is configured to extend up to the alignment mechanism such that the retractable interchangeable blade abuts the alignment mechanism.

In a non-limiting exemplary embodiment, the interchangeable retractable blades includes a first interchangeable retractable blade including a distal end having a radius of curvature substantially equal to a radius of curvature of the external guide, a second interchangeable retractable blade including a distal end having a radius of curvature substantially less than a radius of curvature of the external guide, and a third interchangeable retractable blade including opposed longitudinal beveled-edges.

The present disclosure further includes a method of utilizing a precision blade apparatus for precisely severing a target object including at least one of a vein, an artery, a capillary, a nerve muscle, and an organ during a medical surgical procedure. Such a method includes the steps of: providing a portable, hand-held housing; providing and attaching a control mechanism to the housing; providing a blade motor and a power source, each contained within the housing; communicating each of the blade motor and the power source with the control mechanism; providing and locating an adjustment dial at a proximal end of the housing; operably coupling the adjustment dial to the blade motor; providing and operably coupling a retractable interchangeable blade to the blade motor such that the retractable interchangeable blade is configured to be operated by the control mechanism and extended outwardly from a distal end of the housing; providing and attaching an alignment mechanism to an outer surface of the housing such that the alignment mechanism is operated by the control mechanism; and allowing more nuanced control and illumination of a particular area of the target object by providing and attaching a secondary alignment mechanism to the alignment mechanism. Such a secondary alignment mechanism includes a series of light-emitting diodes configured to be aligned into a single point when the target object is appropriately centered within the alignment mechanism. The method further includes the step of: calibrating and adjusting, via the adjustment dial, a blade extension speed, a blade extension distance, a blade selection of the retractable interchangeable blade, a position of the alignment mechanism, operation of the secondary alignment mechanism, and power consumption.

In a non-limiting exemplary embodiment, the method further including the steps of: prior to beginning the medical surgical procedure, selecting a desired one of the blade selection and a desired one of the blade extension speed; the alignment mechanism collecting the target object, wherein the target object is a single artery; the secondary alignment mechanism indicating a topography and a depth of the single artery; a user assessing a cut depth necessary for each individual layer of the single artery; prior to a cutting the single artery, adjusting the adjustment dial to control the alignment mechanism to a desired position; and while cutting the single artery, adjusting the adjustment dial to control the extension of the retractable blade.

In a non-limiting exemplary embodiment, the method further including the steps of: prior to beginning the medical surgical procedure, selecting a desired one of the blade selection and a desired one of the blade extension speed; the alignment mechanism collecting the target object, wherein the target object is a bundle of nerves; the secondary alignment mechanism indicating a topography and a depth of each individual nerve in the bundle of nerves; a user assessing a cut depth necessary for each individual nerve in the bundle of nerves; prior to a cutting each individual nerve in the bundle of nerves, adjusting the adjustment dial to control the alignment mechanism to a desired position; and while individually cutting each individual nerve in the bundle of nerves, adjusting the adjustment dial to control the extension of the retractable blade.

There has thus been outlined, rather broadly, the more important features of non-limiting exemplary embodiment(s) of the present disclosure so that the following detailed description may be better understood, and that the present contribution to the relevant art(s) may be better appreciated. There are additional features of the non-limiting exemplary embodiment(s) of the present disclosure that will be described hereinafter and which will form the subject matter of the claims appended hereto.

### BRIEF DESCRIPTION OF THE NON-LIMITING EXEMPLARY DRAWINGS

The novel features believed to be characteristic of non-limiting exemplary embodiment(s) of the present disclosure are set forth with particularity in the appended claims. The non-limiting exemplary embodiment(s) of the present disclosure itself, however, both as to its organization and method of operation, together with further objects and advantages thereof, may best be understood by reference to the following description taken in connection with the accompanying drawings in which:

FIG. 1 illustrates an exemplary precision blade apparatus, according to some embodiments of the present disclosure;

FIG. 2A illustrates an alternate view of an exemplary precision blade apparatus, wherein the housing is shown in a transparent view;

FIG. 2B illustrates an alternate view of an exemplary precision blade apparatus, wherein the housing is shown in a transparent view FIG. 2C illustrates an alternate view of an exemplary precision blade apparatus, wherein the housing is shown in a transparent view;

FIG. 3A illustrates an alternate view of an exemplary precision blade apparatus, wherein the alignment mechanism is in a released position;

FIG. 3B illustrates an alternate view of an exemplary precision blade apparatus, wherein the alignment mechanism is in a released position;

FIG. 3C illustrates an alternate view of an exemplary precision blade apparatus, wherein the alignment mechanism is in a released position;

FIG. 4 illustrates an exemplary precision blade apparatus with removable head housing and pivoting neck, wherein the housing is shown in transparent view;

FIG. 5A illustrates an exemplary precision blade apparatus with a pivoting neck and removable head housing;

FIG. 5B illustrates an exemplary precision blade apparatus with a pivoting neck and removable head housing;

FIG. 6 illustrates an exemplary precision blade apparatus with an exploded alignment mechanism, according to some embodiments of the present disclosure;

FIG. 7 illustrates an exemplary precision blade apparatus with a blade extended, according to some embodiments of the present disclosure;

FIG. 8A illustrates an exemplary precision blade apparatus with a large interchangeable retractable blade, according to some embodiments of the present disclosure;

FIG. 8B illustrates an exemplary precision blade apparatus with a medium interchangeable retractable blade, according to some embodiments of the present disclosure;

FIG. 8C illustrates an exemplary precision blade apparatus with a small interchangeable retractable blade, according to some embodiments of the present disclosure;

FIG. 8D illustrates an exemplary precision blade apparatus with a medium beveled-edge interchangeable retractable blade, according to some embodiments of the present disclosure; and

FIG. 8E illustrates an exemplary precision blade apparatus with a small beveled-edge interchangeable retractable blade, according to some embodiments of the present disclosure.

Those skilled in the art will appreciate that the figures are not intended to be drawn to any particular scale; nor are the figures intended to illustrate every non-limiting exemplary embodiment(s) of the present disclosure. The present disclosure is not limited to any particular non-limiting exemplary embodiment(s) depicted in the figures nor the shapes, relative sizes or proportions shown in the figures.

### DETAILED DESCRIPTION OF NON-LIMITING EXEMPLARY EMBODIMENT(S) OF THE PRESENT DISCLOSURE

The present disclosure will now be described more fully hereinafter with reference to the accompanying drawings, in which non-limiting exemplary embodiment(s) of the present disclosure is shown. The present disclosure may, however, be embodied in many different forms and should not be construed as limited to the non-limiting exemplary embodiment(s) set forth herein. Rather, such non-limiting exemplary embodiment(s) are provided so that this application will be thorough and complete, and will fully convey the true spirit and scope of the present disclosure to those skilled in the relevant art(s). Like numbers refer to like elements throughout the figures.

The illustrations of the non-limiting exemplary embodiment(s) described herein are intended to provide a general understanding of the structure of the present disclosure. The illustrations are not intended to serve as a complete description of all of the elements and features of the structures, systems and/or methods described herein. Other non-limiting exemplary embodiment(s) may be apparent to those of ordinary skill in the relevant art(s) upon reviewing the disclosure. Other non-limiting exemplary embodiment(s) may be utilized and derived from the disclosure such that structural, logical substitutions and changes may be made without departing from the true spirit and scope of the present disclosure. Additionally, the illustrations are merely representational are to be regarded as illustrative rather than restrictive.

One or more embodiment(s) of the disclosure may be referred to herein, individually and/or collectively, by the term “non-limiting exemplary embodiment(s)” merely for convenience and without intending to voluntarily limit the true spirit and scope of this application to any particular non-limiting exemplary embodiment(s) or inventive concept. Moreover, although specific embodiment(s) have been illustrated and described herein, it should be appreciated that any subsequent arrangement designed to achieve the same or similar purpose may be substituted for the specific embodiment(s) shown. This disclosure is intended to cover any and all subsequent adaptations or variations of other embodiment(s). Combinations of the above embodiment(s), and other embodiment(s) not specifically described herein, will be apparent to those of skill in the relevant art(s) upon reviewing the description.

References in the specification to “one embodiment(s)”, “an embodiment(s)”, “a preferred embodiment(s)”, “an alternative embodiment(s)” and similar phrases mean that a particular feature, structure, or characteristic described in connection with the embodiment(s) is included in at least an embodiment(s) of the non-limiting exemplary embodiment(s). The appearances of the phrase “non-limiting exemplary embodiment” in various places in the specification are not necessarily all meant to refer to the same embodiment(s).

Directional and/or relationary terms such as, but not limited to, left, right, nadir, apex, top, bottom, vertical, horizontal, back, front and lateral are relative to each other and are dependent on the specific orientation of an applicable element or article, and are used accordingly to aid in the description of the various embodiment(s) and are not necessarily intended to be construed as limiting.

If used herein, “about” means approximately or nearly and in the context of a numerical value or range set forth means±15% of the numerical.

If used herein, “substantially” means largely if not wholly that which is specified but so close that the difference is insignificant.

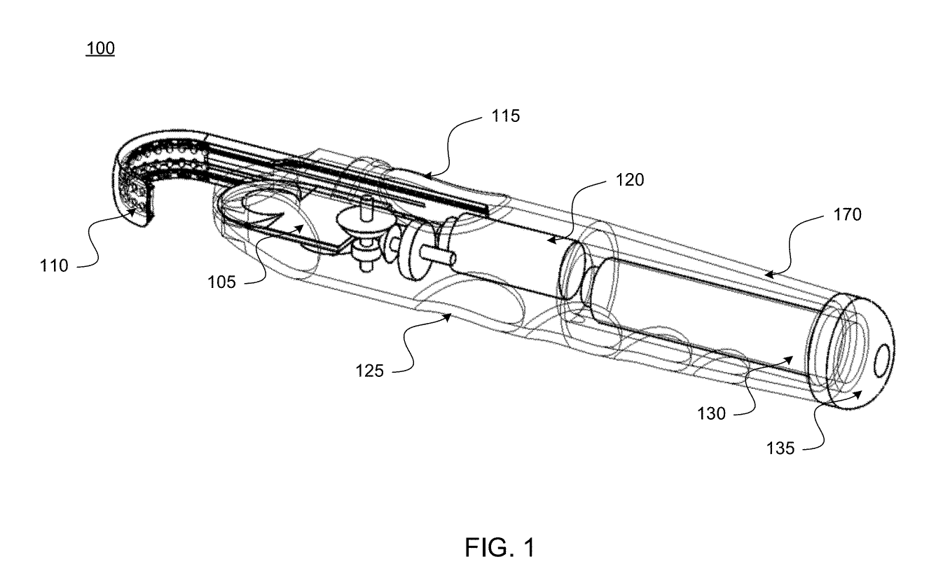

The non-limiting exemplary embodiment(s) is/are referred to generally in FIGS. 1-8E and is/are intended to provide a surgical tool with increased precision. More specifically, the present disclosure relates to a blade apparatus that may allow a surgeon greater control and precision during a procedure by providing a streamlined grip and cutting tool.

In the following sections, detailed descriptions of examples and methods of the disclosure will be given. The description of both preferred and alternative examples, though thorough, are exemplary only, and it is understood to those skilled in the art that variations, modifications, and alterations may be apparent. It is therefore to be understood that the examples do not limit the broadness of the aspects of the underlying disclosure as defined by the claims.

Referring now to FIG. 1, an exemplary precision blade apparatus 100 is illustrated, wherein the housing 170 is shown in a transparent view. In some embodiments, a precision blade apparatus 100 may comprise a retractable blade 105 that may extend from the housing 170 utilizing a blade motor 120, wherein the extension may be based in part on input from a control mechanism 115. In some implementations, the precision blade apparatus 100 may comprise a power source 130, which may provide power to the blade motor 120 and other electronic functionality, such as an alignment mechanism 110. In some aspects, the power source 130 may be replaceable, such as a battery. In some embodiments, the power source 130 may be rechargeable, wherein the precision blade apparatus 100 may further comprise a charging port, such as a micro USB port.

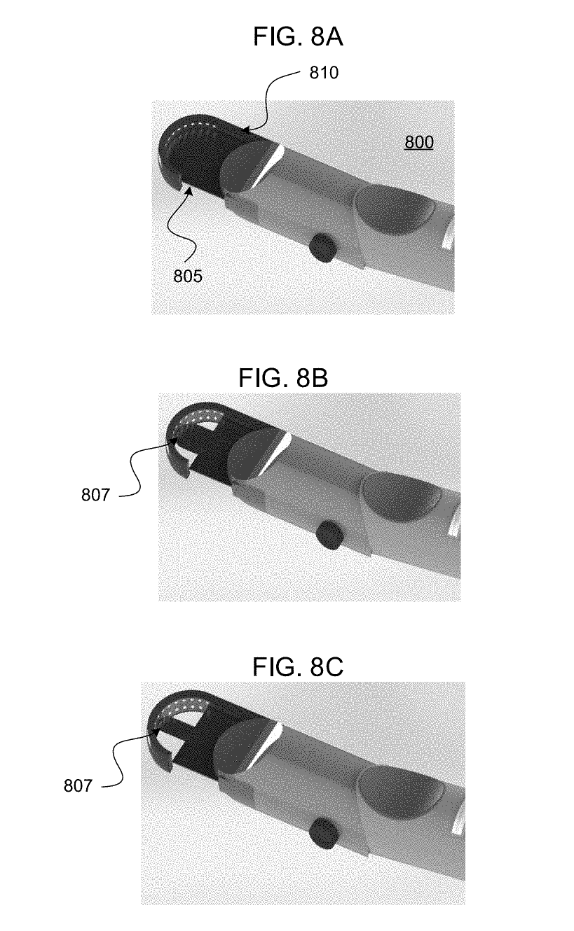

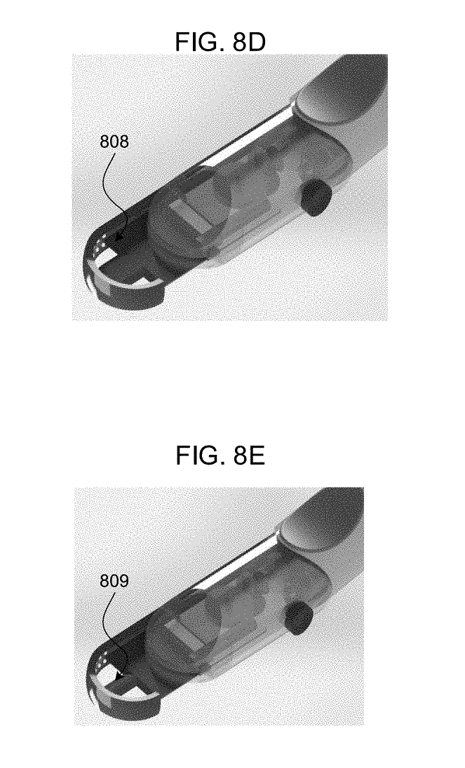

In some aspects, the retractable blade 105 may be interchangeable, wherein different size retractable blades 105 may be used in the same precision blade apparatus 100, such as illustrated in FIGS. 8A-8E. In some embodiments, the housing 170 may hold one retractable blade 105 at a time, wherein the blade size may be manually interchanged. In some aspects, the housing 170 may hold a plurality of retractable blades 105, wherein the blade size may be selected from the inserted sizes through one or both the control mechanism 115 and an adjustment dial 135.

In some aspects, the housing 170 may comprise a gripping mechanism 125 that may allow for a secure hold of the precision blade apparatus 100 during use. In some embodiments, the gripping mechanism 125 may comprise finger divots that may limit movement of a user's hand. In some implementations, the gripping mechanism 125 may comprise one or both a textured surface, such as hatching or beading, or an anti-slippage material, such as rubber or silicone.

In some embodiments, the precision blade apparatus 100 may comprise an alignment mechanism 110, which may allow a user to accurately and precisely align the retractable blade 105 with a target object. In some aspects, the alignment mechanism 110 may comprise a curving arm that may be controlled to collect and stabilize a target object. In some embodiments, the alignment mechanism 110 may comprise a secondary alignment mechanism that may allow for more nuanced control. For example, the secondary alignment mechanism may comprise a series of LED lights that may illuminate the target object. In some implementations, the secondary alignment mechanism may allow a user to target a particular area on the target object. For example, the LED lights may align into a single point when the target object is appropriately centered within the alignment mechanism 110.

In some aspects, the target may comprise a single object, such as a vein, artery, capillary, or nerve. In some embodiments, the target may comprise a group of objects, such as a bundle of capillaries or nerves. In some implementations, the target may comprise a portion of an object, such as a layer of muscle, organ, nerve, vein, or artery. In some embodiments, an alignment mechanism 110 may allow for alignment of a range of targets.

For example, an alignment mechanism 110 may collect a bundle of nerves, and a secondary alignment mechanism, such as a series of LED lights, may allow a user to target each nerve individually, wherein each cut may sever a single nerve. As another example, an alignment mechanism 110 may collect a single artery, and a secondary alignment mechanism may indicate topography and depth, which may allow a user to assess the cut depth necessary for each individual layer of the artery.

In some implementations, the precision blade apparatus 100 may comprise an adjustment dial 135, which may be located at the base or distal end of the housing 170, wherein the adjustment dial 135 may control a predefined set of functionality, such as blade extension speed, blade extension distance, or blade selection as non-limiting examples. In some aspects, the control mechanism 115 may comprise a single button wherein progressive pressing may trigger different actions. In some implementations (not shown), the control mechanism 115 may comprise a series of buttons, wherein each button may control a unique function of the precision blade apparatus 100.

In some aspects (not shown), one or both the functionality of the control mechanism 115 and the adjustment dial 135 may be integrated into a touch screen that may allow for control of one or more functions of the precision blade apparatus 100. In some embodiments, the functionality of the control mechanism 115 and the adjustment dial 135 may comprise a combination of buttons and touchscreen. In some aspects, some of the functionality may be preprogrammed prior to a procedure and some functionality may be controlled during the procedure.

As an illustrative example, a touchscreen may allow for preprogramming prior to a procedure, such as selecting the blade sizes, base blade extension speed, control preferences, and alignment preferences, as non-limiting examples. The control mechanism 115 may allow for control of some functionality during the procedure. For example, the control mechanism 115 may allow for complete control over blade extension and nuanced control over the blade extension speed within a predefined range based on the preprogrammed base blade extension speed.

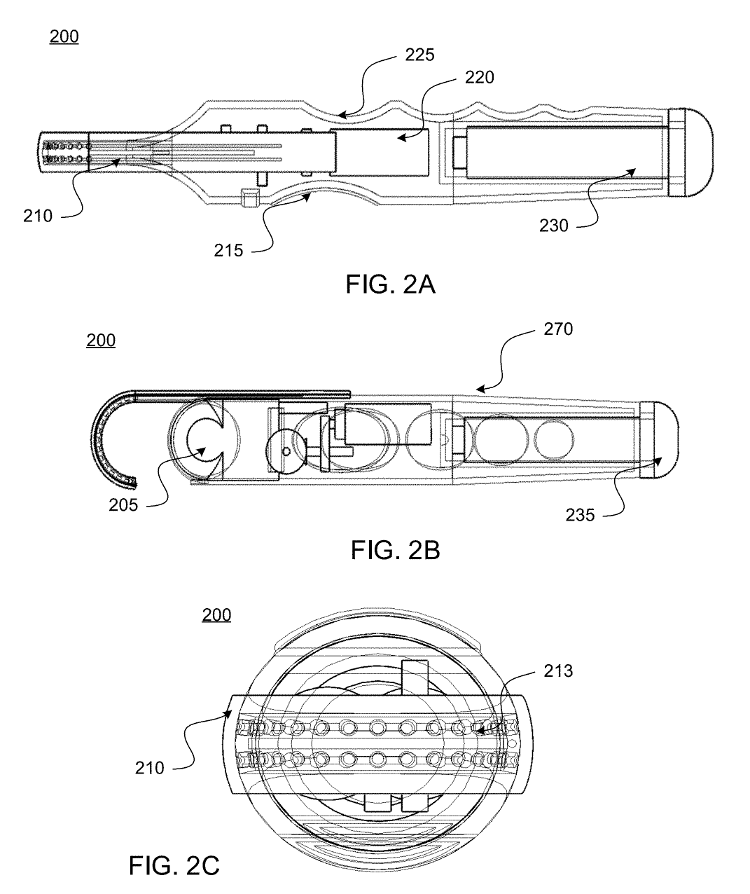

Referring now to FIGS. 2A-2C, alternate views of an exemplary precision blade apparatus 200 are illustrated, wherein the housing 270 is shown in a transparent view. In some aspects, a precision blade apparatus 200 may comprise a housing 270, which may contain a power source 230, blade motor 220, and retractable blade 205. In some embodiments, a control mechanism 215 may comprise a button that may engage with the blade motor 220, wherein engagement may trigger a blade motor 220 action, such as extension or retraction of the retractable blade 205.

In some aspects, a precision blade apparatus 200 may comprise a blade insertion mechanism, which may allow for safe and easy insertion and removal of retractable blades 205. In some implementations, the blade insertion mechanism may allow a user to place a retractable blade 205 proximate to a blade pocket and the blade insertion mechanism may grab and accept the retractable blade 205 into the blade pocket, without or with limited force by the user. In some aspects, the blade insertion mechanism may allow for easy and safe ejection and removal of a retractable blade 205.

In some embodiments, the precision blade apparatus 200 may comprise an alignment mechanism 210, which may attach to the exterior of the housing 270. In some implementations, the alignment mechanism 210 may be detachable and interchangeable, which may allow for different alignment mechanism 210 types on a precision blade apparatus 200. Different alignment mechanisms 210 may be useful for different applications. For example, completely severing a target object may benefit from a different alignment mechanism 210 than may be useful for cutting through a single layer within a target object. In some aspects, the alignment mechanism 210 may comprise a secondary alignment mechanism 213, such as rows of LED lights that may illuminate the target object. In some embodiments, illumination may allow for easy viewing of the operating area.

In some aspects, the precision blade apparatus 200 may comprise an alignment sensor that may ensure that the alignment mechanism 210 may close effectively. In some embodiments, the alignment sensor may ensure that the alignment mechanism 210 may provide accurate alignment indicators relative to the retractable blade 205. In some implementations, the alignment sensor may provide an indication of when the alignment mechanism 210 is appropriately aligned, such as a tactile, auditory, or visual indication. For example, the alignment sensor may turn green when properly aligned. As another example, the alignment sensor may beep once when properly aligned.

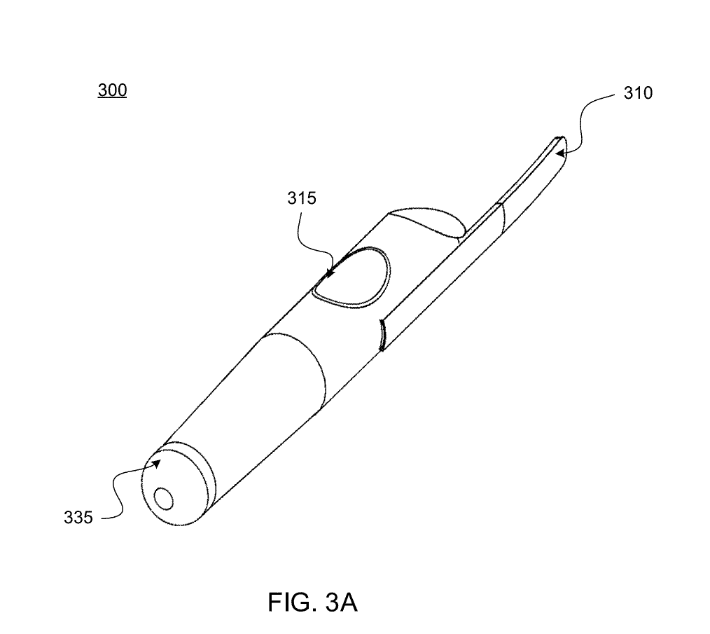

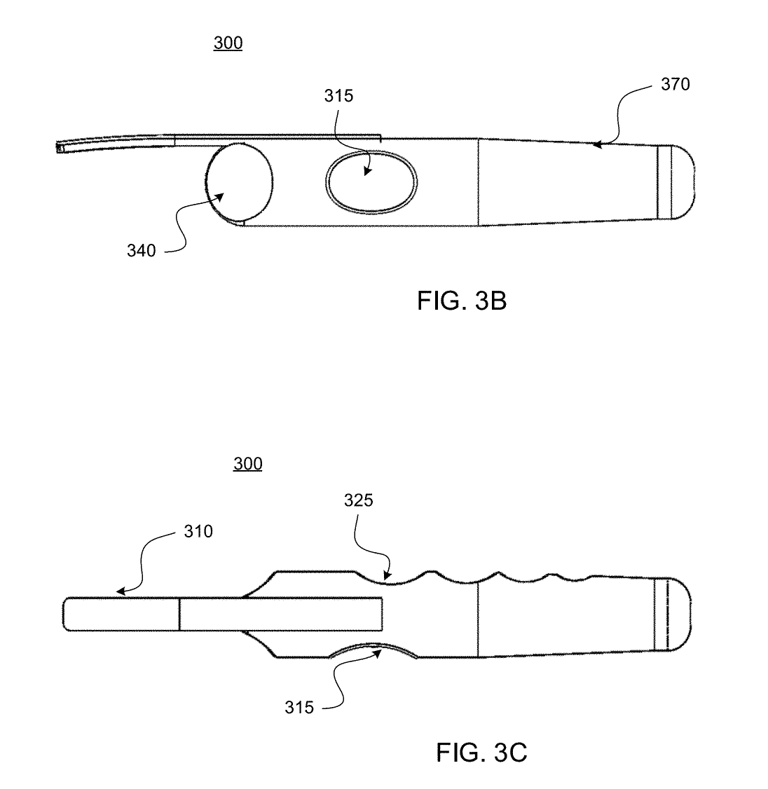

Referring now to FIGS. 3A-3C, alternate views of an exemplary precision blade apparatus 300 are illustrated, wherein the alignment mechanism 310 is in a released position. In some aspects, a precision blade apparatus 300 may comprise a housing 370 with a gripping mechanism 325, which may limit the risk of the precision blade apparatus 300 shifting within the user's hands during a procedure. In some embodiments, the housing 370 may comprise a blade pocket 340 that may secure a retractable blade (not shown) when not in use. In some implementations, the blade pocket 340 may comprise tapering walls to limit movement of the retractable blade when secured within the blade pocket 340.

Securing the retractable blade when not in use may provide multiple benefits. For example, uncontrolled shifting of the retractable blade may reduce the accuracy when the retractable blade is extended. As another example, movement of the retractable blade may affect the quality of the retractable blade, increasing the chance for dulling or chipping when the precision blade apparatus 300 is not in use.

In some aspects, the housing 370 may comprise multiple parts, wherein at least a portion of the parts may be removable. For example, a portion of the housing 370 may be removed to expose a power source, which may allow for replacement of the power source. As another example, a portion of the blade pocket 340 may be removable or adjustable, which may allow for removal and replacement of the retractable blades. Over time, a retractable blade may become less effective or may become damaged, wherein replacement with a new retractable blade may be necessary. In some aspects, retractable blades may be interchangeable based on the procedure and target object.

In some embodiments, the precision blade apparatus 300 may comprise an adjustment dial 335, which may allow for calibration and adjustment of one or more functionality and characteristics of the precision blade apparatus 300. As a non-limiting example, the adjustment dial 335 may allow for control of the blade extension speed, blade extension length, blade selection, alignment mechanism position, secondary alignment mechanism, and general power. In some aspects, the adjustment dial 335 may allow a user to select what functionality they wish to control, and the control mechanism 315 may be used to control the selected functionality.

As an illustrative example, prior to the procedure, a user may toggle between blade selection and blade extension speed to select the blade size and base blade extension speed. Prior to a cut, a user may toggle the adjustment dial 335 to control the alignment mechanism 310. During the cut, a user may toggle the adjustment dial 335 to control the extension of the retractable blade.

In some aspects, the control mechanism 315 may comprise a single button that may trigger a series of actions. For example, clicking the control mechanism 315 may allow a user to toggle between functionality, and holding the control mechanism 315 may control the selected functionality. In some embodiments, the precision blade apparatus 300 may comprise a control indicator that may show which functionality the control mechanism 315 is currently engaging, which may reduce accidentally engaging an unintended functionality. In some implementations, the control indicator may comprise a series of LED lights, wherein the color or location of the illuminated light may indicate the engaged functionality.

Though shown as a dial located at a distal end of the precision blade apparatus 300, other embodiments may be appropriate. In some aspects, the adjustment dial 335 may comprise a series of buttons, such as along the gripping mechanism 325, which may allow for toggling between functionality and control without having to use two hands or without having to move the guiding hand.

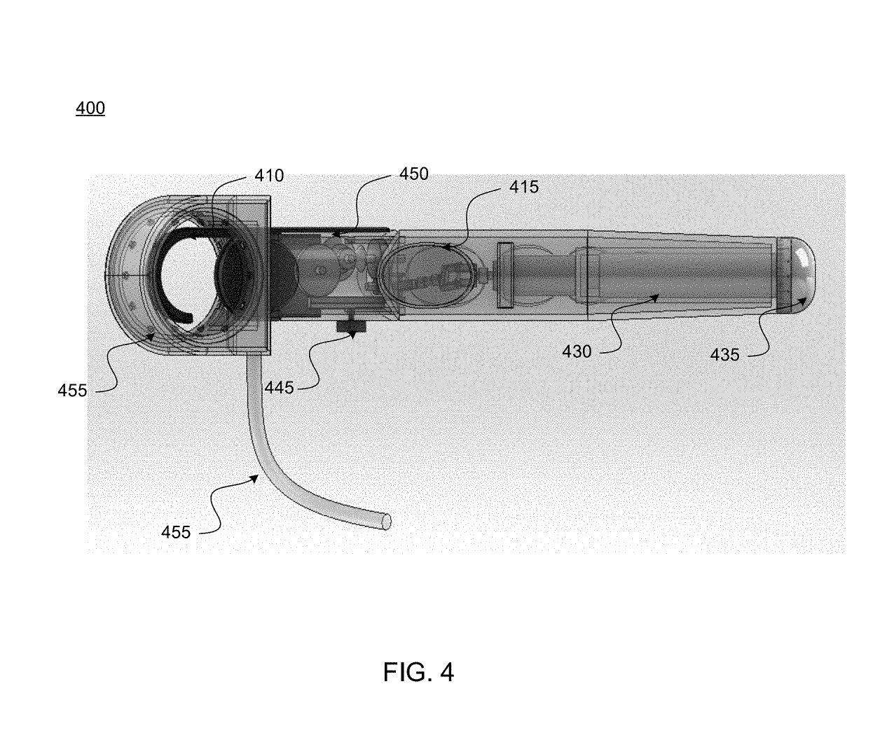

Referring now to FIG. 4, an exemplary precision blade apparatus 400 with removable head housing 455 and pivoting neck 450 is illustrated, wherein the removable head housing 455 is shown in transparent view. In some aspects, a precision blade apparatus 400 may comprise a control mechanism 415, a power source 430, an adjustment dial 435, and an alignment mechanism 410, such as illustrated and described in FIG. 1. In some embodiments, the precision blade apparatus 400 may comprise a pivoting neck 450, which may allow for more comfortable operation of the precision blade apparatus. In some aspects, a pivoting neck 450 may allow for adjustment through a pivot control mechanism 445 of the angle of approach for the cut without requiring a user to strain their hand to appropriately position the precision blade apparatus.

In some implementations, the precision blade apparatus 400 may comprise a removable head housing 455, which may allow for control of the micro environment surrounding the target object. In some embodiments, the removable head housing 455 may be slipped over the alignment mechanism 410 once the target object may be collected and before the cut. In some aspects, the removable head housing 455 may be operated in conjunction with or similarly to the alignment mechanism 410, wherein the removable head housing 455 may coil around a collected target object.

In some embodiments, the removable head housing 455 may comprise one or more mechanisms configured to control the environment surrounding the target objects. For example, the walls may be prefilled with the environment control chemicals that may be periodically released during the procedure to ensure a stable environment. As another example, the removable head housing 455 may comprise solid state thermal plates, which may allow for precise temperature control of the environment.

In some aspects, the removable head housing 455 may comprise an environment control tube 455 that may pump in environment control chemicals throughout the procedure. In some implementations, an environment control tube 455 may allow for more sustained and more variable control of the environment in contrast to embodiments where the removable head housing 455 may be prefilled. The environment control tube 455 may require connection to an external device that may provide the necessary environment control chemicals.

In some aspects, it may be necessary to control the temperature within the environment. In some embodiments, significantly reducing the temperature within the ambient environment may provide a range of benefits. For example, lowered temperatures may reduce the risk of a target object receding once severed. In some implementations, clamping both sides of the target object may also reduce the risk of receding, but in procedures where the target object is particularly small, or clamps may not be practical, lowered temperatures may mitigate that risk. As another example, lowering the temperature may slow blood flow, limiting the loss of blood from a cut. In some aspects, loss of blood may reduce visibility of the target object and may require cleaning of the area and repositioning of the precision blade apparatus.

In some aspects, flowing particular chemicals over a surgical site may provide a range of benefits, such as a reduction in risk for infection and slowed blood flow. For example, carbon dioxide, nitrogen, and nitric oxide may be flowed through the micro environment within the removable head housing 455 throughout the procedure.

In some aspects, one or both the alignment mechanism 410 and the removable head housing 455 may further comprise sensors, which may monitor one or both the micro environment and the exterior environment. In some aspects, the sensors may monitor the air content within the micro environment, which may indicate whether more or less chemicals may need to be flowed into the environment. In some embodiments, the sensors may monitor temperature of one or more objects, such as the ambient air within the environment, the external surface of the target object, the exposed surface of the target object as it is cut, the blade, or other relevant objects within the environment.

In some implementations, the relative temperature of the blade to the target object may be significant, wherein control of the temperature of the blade may be necessary or useful. In some embodiments, the temperature of the retractable blade may be controlled. For example, the temperature of the retractable blade may be at least partially controlled while the retractable blade is contained within the blade pocket, and then monitored as the retractable blade is extended into the environment. As another example, the temperature of the retractable may be at least partially controlled by one or both the alignment mechanism 410 and the removable head housing 455, which may target the surface of the retractable blade or the portion of the environment directly outside the blade pocket.

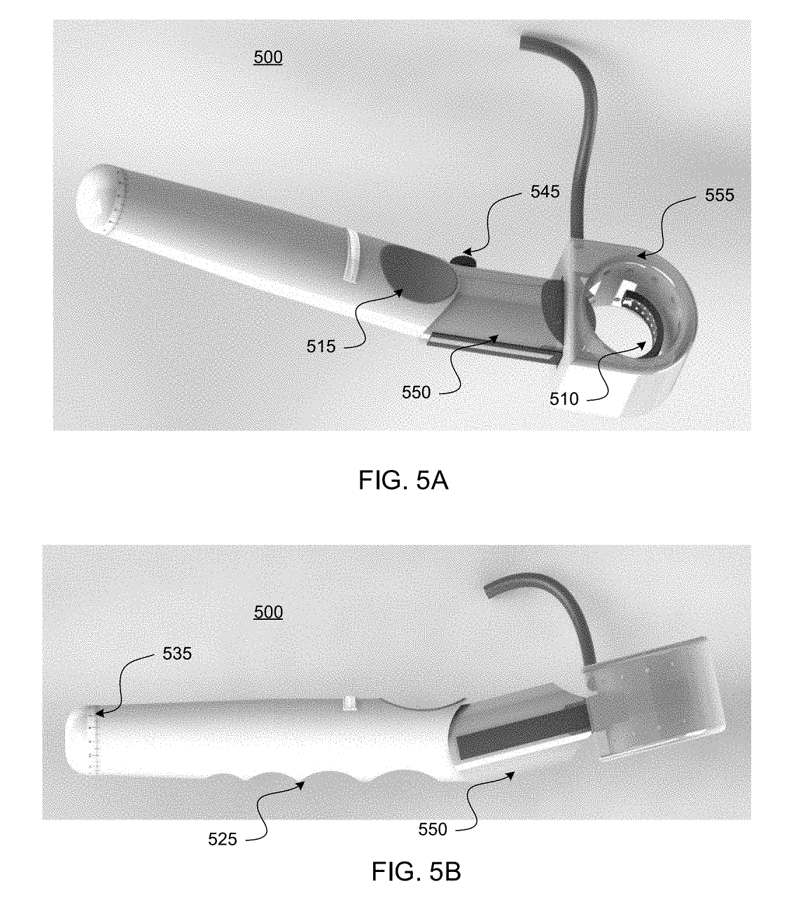

Referring now to FIGS. 5A-5B, an exemplary precision blade apparatus 500 with a pivoting neck 550 and removable head housing 555 is illustrated. In some aspects, a precision blade apparatus 500 may comprise a removable head housing 555, which may be configured to surround one or more the target object, an alignment mechanism 510, and the retractable blade. In some implementations, the removable head housing 555 may surround a blade pocket, such an illustrated in FIG. 3B, which may allow for some control of characteristics of one or more the blade, the environment within the blade pocket, and the environment directly outside the blade pocket.

In some aspects, a precision blade apparatus 500 may comprise a pivoting neck 550, which may allow a user to adjust the angle of the head relative to the gripping mechanism 525. In some embodiments, the angle of the pivoting neck 550 may be operated by a separate pivot control mechanism 545, which may be located near the primary control mechanism 515. In some implementations, the pivot control mechanism 545 may be integrated with one or both the primary control mechanism 515 and the adjustment dial 535, such as illustrated and described in FIG. 1.

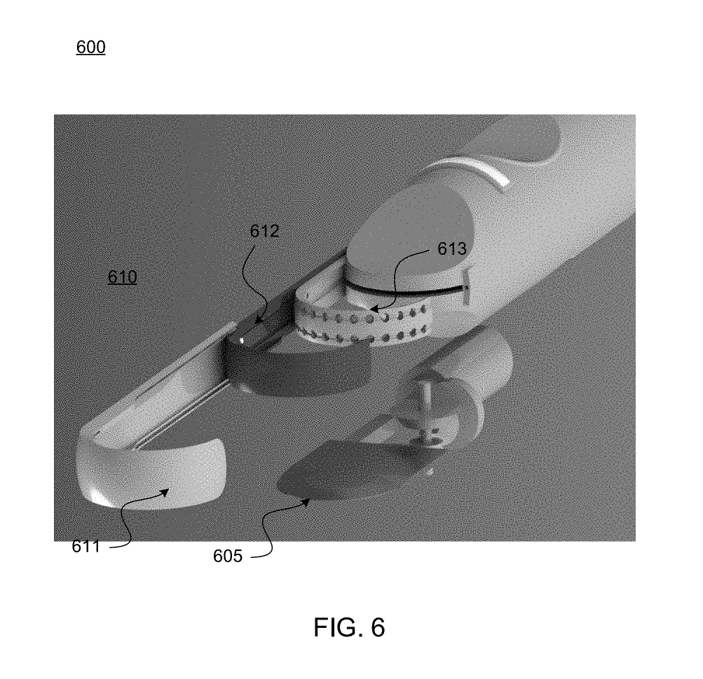

Referring now to FIG. 6, an exemplary precision blade apparatus 600 with an exploded alignment mechanism 610 is illustrated. In some aspects, the alignment mechanism 610 may comprise an external guide 611, and internal guide 612, and a secondary alignment mechanism 613, such as described in FIG. 1. In some aspects, the secondary alignment mechanism 613 may attach to the internal guide 612, which may be secured to the external guide 611.

In some implementations, one or both the external guide 611 and the internal guide 612 may be directly controlled through the precision blade apparatus. In some embodiments, the alignment mechanism 610 may be retracted and collapse around the surface of the blade pocket while the precision blade apparatus 600 is not in use, which may protect one or more the alignment mechanism 610, the retractable blade 605, and the blade pocket. For example, wrapping the alignment mechanism 610 around the opening of the blade pocket may limit the risk of contamination of the retractable blade 605.

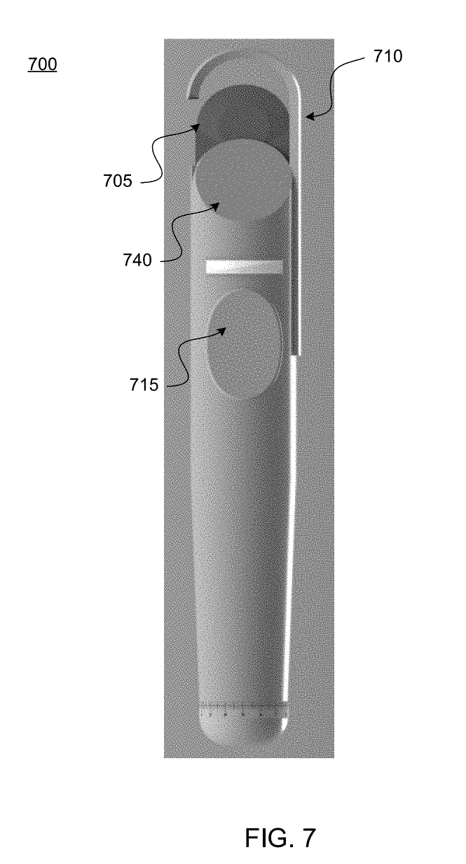

Referring now to FIG. 7, an exemplary precision blade apparatus 700 with a blade 705 extended is illustrated. In some aspects, a retractable blade 705 of a precision blade apparatus 700 may extend from a blade pocket 740. In some implementations, the extension of the retractable blade 705 may be operated by a control mechanism 715. In some embodiments, the retractable blade 705 may extend up to the alignment mechanism 710, wherein the retractable blade 705 may abut the alignment mechanism 710. In some aspects, it may not be necessary or useful to extend that far, and a user may be able to control the precise extension distance.

Referring now to FIGS. 8A-8E, an exemplary precision blade apparatus 800 is illustrated with a range of interchangeable retractable blades 805-809. In some aspects, the precision blade apparatus 800 may be adapted for a range of applications, wherein different applications may utilize different retractable blades 805-809. In some embodiments, the precision blade apparatus 800 may contain a single retractable blade 805-809 at a time, wherein the different retractable blades 805-809 may be removed and installed between applications.

In some embodiments, the precision blade apparatus 800 may comprise an internal sterilization mechanism, which may allow for some sterilization of the retractable blades 805-809 between use. In some aspects, one or more the components of the precision blade apparatus 800 may be sterilized prior to or after use. In some embodiments, the components may be sterilized according to standard methods, such as chemical, heat, or other methods. In some implementations, different components may allow for different types of sterilization. For example, some components may be disposable, some components may be autoclaved, and others may be sprayed with a sterilizing chemical.

In some implementations, the precision blade apparatus 800 may contain multiple retractable blades 805-809 at a time, allowing a user to control which retractable blade 805-809 may be extended. Containing multiple retractable blades 805-809 may be preferable for applications where different retractable blade 805-809 types and sizes may be utilized for the same procedure, reducing the need to stop the procedure to manually exchange the retractable blade 805-809.

As an illustrative example, a large retractable blade 805 may be preferable when the procedure requires severance of a single target object, wherein the alignment mechanism 810 may be able to collect the individual target object without collecting proximate objects. The medium blades 806, 808 may be preferable when the target object may not consume the entire space within the alignment mechanism 810. The small blades 807, 809 may be preferable when the procedure requires severance of a single target object, wherein the alignment mechanism 810 may collect a bundle of objects and the target object is one within that bundle. Beveled-edge blades 808, 809 may be preferable when a procedure requires a slit or a cut into a portion of the target object.

While non-limiting exemplary embodiment(s) has/have been described with respect to certain specific embodiment(s), it will be appreciated that many modifications and changes may be made by those of ordinary skill in the relevant art(s) without departing from the true spirit and scope of the present disclosure. It is intended, therefore, by the appended claims to cover all such modifications and changes that fall within the true spirit and scope of the present disclosure. In particular, with respect to the above description, it is to be realized that the optimum dimensional relationships for the parts of the non-limiting exemplary embodiment(s) may include variations in size, materials, shape, form, function and manner of operation.

The Abstract of the Disclosure is provided to comply with 37 C.F.R. § 1.72(b) and is submitted with the understanding that it will not be used to interpret or limit the scope or meaning of the claims. In addition, in the above Detailed Description, various features may have been grouped together or described in a single embodiment for the purpose of streamlining the disclosure. This disclosure is not to be interpreted as reflecting an intention that the claimed embodiment(s) require more features than are expressly recited in each claim. Rather, as the following claims reflect, inventive subject matter may be directed to less than all of the features of any of the disclosed non-limiting exemplary embodiment(s). Thus, the following claims are incorporated into the Detailed Description, with each claim standing on its own as defining separately claimed subject matter.

The above disclosed subject matter is to be considered illustrative, and not restrictive, and the appended claims are intended to cover all such modifications, enhancements, and other embodiment(s) which fall within the true spirit and scope of the present disclosure. Thus, to the maximum extent allowed by law, the scope of the present disclosure is to be determined by the broadest permissible interpretation of the following claims and their equivalents, and shall not be restricted or limited by the above detailed description.

What is claimed as new and what is desired to secure by Letters Patent of the United States is:

1. A precision blade apparatus for precisely severing a target object including at least one of a vein, an artery, a capillary, a nerve muscle, and an organ during a medical surgical procedure, said precision blade apparatus comprising:
a housing;
a control mechanism attached to said housing;
a blade motor and a power source each in communication with said control mechanism and contained within said housing;
an adjustment dial located at a proximal end of said housing and being operably coupled to said blade motor;
a retractable interchangeable blade operably coupled to said blade motor and configured to be operated by said control mechanism and extended outwardly from a distal end of said housing;
an alignment mechanism attached to an outer surface of said housing and operated by said control mechanism; and
a secondary alignment mechanism attached to said alignment mechanism, said secondary alignment mechanism including a series of light-emitting diodes configured to be aligned into a single point when the target object is appropriately centered within said alignment mechanism thereby allowing more nuanced control and illumination of a particular area of the target object;
wherein said adjustment dial is configured to calibrate and adjust a blade extension speed, a blade extension distance, a blade selection of said retractable interchangeable blade, a position of said alignment mechanism, operation of said secondary alignment mechanism, and power consumption.

2. A precision blade apparatus for precisely severing a target object including at least one of a vein, an artery, a capillary, a nerve muscle, and an organ during a medical surgical procedure, said precision blade apparatus comprising:
a portable, hand-held housing;
a control mechanism attached to said housing;
a blade motor and a power source each in communication with said control mechanism and contained within said housing;
an adjustment dial located at a proximal end of said housing and being operably coupled to said blade motor;
a retractable interchangeable blade operably coupled to said blade motor and configured to be operated by said control mechanism and extended outwardly from a distal end of said housing;
an alignment mechanism attached to an outer surface of said housing and operated by said control mechanism; and
a secondary alignment mechanism attached to said alignment mechanism, said secondary alignment mechanism including a series of light-emitting diodes configured to be aligned into a single point when the target object is appropriately centered within said alignment mechanism thereby allowing more nuanced control and illumination of a particular area of the target object;
wherein said adjustment dial is configured to calibrate and adjust a blade extension speed, a blade extension distance, a blade selection of said retractable interchangeable blade, a position of said alignment mechanism, operation of said secondary alignment mechanism, and power consumption.

3. The precision blade apparatus of claim 2, wherein said retractable interchangeable blade comprises: a plurality of uniquely sized interchangeable retractable blades.

4. The precision blade apparatus of claim 2, wherein said housing further comprises: a gripping mechanism including finger divots configured to limit movement of a user hand, said gripping mechanism including a textured surface and an anti-slippage material.

5. The precision blade apparatus of claim 2, wherein said alignment mechanism comprises: a curving arm configured to accurately and precisely align said retractable blade with the target object, said alignment mechanism being configured to collect and stabilize the target object.

6. The precision blade apparatus of claim 2, wherein said control mechanism comprises:
a plurality of buttons configured to maintain control over said blade extension and nuanced control over said blade extension speed within a predefined range based on a predetermined base blade extension speed.

7. The precision blade apparatus of claim 6, wherein one of said buttons is configured to trigger said blade motor to extend and retract said retractable interchangeable blade.

8. The precision blade apparatus of claim 2, further comprising:
a blade pocket located at the distal end of said housing; and
a blade insertion mechanism located at the distal end of said housing and configured to received said retractable interchangeable blade proximate to said blade pocket such that said blade insertion mechanism grabs, accepts, and ejects said retractable interchangeable blade relative to said blade pocket.

9. The precision blade apparatus of claim 2, wherein the alignment mechanism is configured to be toggled between a released position and an engaged position such that said alignment mechanism is detachably and interchangeably attached to an exterior of the housing.

10. The precision blade apparatus of claim 8, wherein said blade pocket comprises:
a plurality of tapering walls configured to limit movement of said retractable interchangeable blade when secured within said blade pocket.

11. The precision blade apparatus of claim 2, further comprising:
a pivot control mechanism operably coupled to said control mechanism;
a removable head housing operably coupled to said pivot control mechanism; and
a pivoting neck operably coupled to said pivot control mechanism to pivot to a desired cut angle without straining a user hand;
wherein said removable head housing is configured to be slipped over the alignment mechanism once the target object is collected and before the cutting procedure;
wherein said removable head housing is configured to coil around the target object.

12. The precision blade apparatus of claim 11, wherein said removable head housing comprises:
a plurality of walls prefilled with environment control chemicals that are periodically released during the medical surgical procedure to lower an environmental temperature and slow blood flow at the target object;
a plurality of solid-state thermal plates configured to precisely control the environmental temperature at the target object; and
an environment control tube configured to pump in the environment control chemicals throughout the medical surgical procedure and thereby lower the environmental temperature at the target object.

13. The precision blade apparatus of claim 12, wherein the environment control chemicals comprises: carbon dioxide, nitrogen, and nitric oxide configured to flow through said removable head housing;
wherein said alignment mechanism and said removable head housing are configured to control a temperature of a surface of the retractable interchangeable blade;
wherein said pivoting neck is connected to said removable head housing, said removable head housing being configured to surround the target object, said alignment mechanism, said retractable interchangeable blade, and said blade pocket.

14. The precision blade apparatus of claim 13, wherein said pivoting neck is configured to adjust an angle of said removable head housing relative to said gripping mechanism, wherein said angle of said pivoting neck is operably coupled to said pivot control mechanism located adjacent to said control mechanism.

15. The precision blade apparatus of claim 8, wherein said alignment mechanism comprises: an external guide internal guide slidably secured thereto;
wherein said secondary alignment mechanism is attached to said internal guide;
wherein each of said external guide and said internal guide are configured to be directly controlled at said housing;
wherein said alignment mechanism is configured to be retracted and collapsed around a surface opening of said blade pocket as well as said retractable interchangeable blade during non-use conditions.

16. The precision blade apparatus of claim 8, wherein said retractable interchangeable blade is configured to be selectively extended outwardly from said blade pocket and configured to be operated by said control mechanism;
wherein said retractable interchangeable blade is configured to extend up to said alignment mechanism such that said retractable interchangeable blade abuts said alignment mechanism.

17. The precision blade apparatus of claim 15, wherein said interchangeable retractable blades comprises:
a first interchangeable retractable blade including a distal end having a radius of curvature substantially equal to a radius of curvature of said external guide;
a second interchangeable retractable blade including a distal end having a radius of curvature substantially less than a radius of curvature of said external guide; and
a third interchangeable retractable blade including opposed longitudinal beveled-edges.

18. A method of utilizing a precision blade apparatus for precisely severing a target object including at least one of a vein, an artery, a capillary, a nerve muscle, and an organ during a medical surgical procedure, said method comprising the steps of:
providing a portable, hand-held housing;
providing and attaching a control mechanism to said housing;
providing a blade motor and a power source, each contained within said housing;
communicating each of said blade motor and said power source with said control mechanism;
providing and locating an adjustment dial at a proximal end of said housing;
operably coupling said adjustment dial to said blade motor;
providing and operably coupling a retractable interchangeable blade to said blade motor such that said retractable interchangeable blade is configured to be operated by said control mechanism and extended outwardly from a distal end of said housing;
providing and attaching an alignment mechanism to an outer surface of said housing such that said alignment mechanism is operated by said control mechanism;
allowing more nuanced control and illumination of a particular area of the target object by providing and attaching a secondary alignment mechanism to said alignment mechanism, said secondary alignment mechanism including a series of light-emitting diodes configured to be aligned into a single point when the target object is appropriately centered within said alignment mechanism; and
calibrating and adjusting, via said adjustment dial, a blade extension speed, a blade extension distance, a blade selection of said retractable interchangeable blade, a position of said alignment mechanism, operation of said secondary alignment mechanism, and power consumption.

19. The method of claim 18, further comprising the steps of:
prior to beginning the medical surgical procedure, selecting a desired one of the blade selection and a desired one of the blade extension speed;
said alignment mechanism collecting the target object, wherein the target object is a single artery;
said secondary alignment mechanism indicating a topography and a depth of the single artery;
a user assessing a cut depth necessary for each individual layer of the single artery;
prior to a cutting the single artery, adjusting said adjustment dial to control said alignment mechanism to a desired position; and
while cutting the single artery, adjusting said adjustment dial to control the extension of the retractable blade.

20. The method of claim 18, further comprising the steps of:
prior to beginning the medical surgical procedure, selecting a desired one of the blade selection and a desired one of the blade extension speed;
said alignment mechanism collecting the target object, wherein the target object is a bundle of nerves;
said secondary alignment mechanism indicating a topography and a depth of each individual nerve in the bundle of nerves;
a user assessing a cut depth necessary for each individual nerve in the bundle of nerves;
prior to a cutting each individual nerve in the bundle of nerves, adjusting said adjustment dial to control said alignment mechanism to a desired position; and
while individually cutting each individual nerve in the bundle of nerves, adjusting said adjustment dial to control the extension of the retractable blade.
